(19) United States
(12) Patent Application Publication
(10) Pub. No.: US 2010/0298264 A1
(43) Pub. Date: Nov. 25, 2010

(54) BIODEGRADABLE AND WATER-SOLUBLE HEMOSTATIC MATERIAL AND A METHOD FOR PREPARING THE SAME

(75) Inventors: XIAOCHENG ZHOU, GUANGDONG (CN); XINGHUA HUANG, GUANGDONG (CN)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(73) Assignee: HUIZHOU FORYOU MEDICAL DEVICES CO., LTD, GUANGDONG (CN)

(21) Appl. No.: 12/469,666

(22) Filed: May 20, 2009

(51) Int. Cl. A61K 31/717; A61P 7/04

(52) U.S. Cl. 514/57

(57) ABSTRACT

A biodegradable and water-soluble hemostatic material is provided. The hemostatic material comprises an oxidized regenerated cellulose salt having a degree of carboxylic acid oxidation not less than 5%, a degree of etherification of 0.2 to 1.2 and a number average molecular weight of 50,000 to 200,000. The hemostatic material according to the present invention offers improved hemostatic effect, absorbability and operability over existing hemostatic products. A method for preparing the hemostatic material is further provided.

### BACKGROUND OF THE INVENTION

The present invention relates to the field of hemostatic materials, and more particularly to a biodegradable and water-soluble hemostatic material and a method for preparing the same.

Acute hemorrhage is a main cause of traumatic death, thus hemostasis is an important measure for lasting the lives of wounded persons. Total blood volume of a human is 7-8% of body weight and is about 4000 ml. The wounded persons would get a shock state when they had lost approximately 20% of their total blood volume, and would risk their lives when they had lost approximately 40% of their total blood volume. The wounded persons would feel huge mental and physical pain during hemorrhage, and may be infected by some diseases during blood transfusion. Therefore, how to reduce blood loss is a common and essential issue in tissue injury-repair and surgery.

At present, many kinds of hemostatic materials have been developed for various clinical applications, such as carboxymethyl cellulose, oxidized regenerated cellulose, fibrin glue, collagen, chitosan and the like. Carboxymethyl cellulose type hemostatic material is most widely used. The carboxymethyl cellulose type hemostatic material is one kind of polyhydroxy and polycarboxy polysaccharide having strong water absorbability. The hemostatic material can rapidly and intensively absorb water in blood exuding from wound surfaces, thereby increasing the viscosity and the concentration of the blood and reducing the outflow rate of the blood. The hemostatic material exists in form of viscous gel after absorbing water and fills up the wound surfaces and seals ends of capillary vessels, thereby achieving the purpose of physical hemostasia. The gel formed form the hemostatic material can effectively adsorb blood platelets and hemoglobins, thereby promoting formation of local thrombus and hemostasia. Furthermore, negative ions released from the dissolved hemostatic material can activate coagulation factors and promotes formation of thrombin. Plasma fibrinogen can be rapidly transformed into fibrin under catalysis effect of the thrombin, thereby achieving the purpose of physiology hemostasia. However, the carboxymethyl cellulose type hemostatic material belongs to one kind of cellulose and there is not any enzyme which can decompose the cellulose in human body, thus the hemostatic material cannot be degraded into carbon dioxide and water, and can only be transformed into small molecule substance and absorbed by or excluded from human body slowly.

### SUMMARY OF THE INVENTION

The present invention provides a biodegradable and water-soluble hemostatic material and a method for preparing the same.

In accordance with a first aspect of the present invention, a biodegradable and water-soluble hemostatic material comprises an oxidized regenerated cellulose salt having a degree of carboxylic acid oxidation not less than 5%, a degree of etherification of 0.2 to 1.2 and a number average molecular weight of 50,000 to 200,000.

According to an embodiment of the present invention, the hemostatic material is prepared from viscose fibre having a degree of polymerization of 100 to 1,000 through an oxidation process and an etherification process.

According to an embodiment of the present invention, the degree of carboxylic acid oxidation is in a range of 18% to 24%.

According to an embodiment of the present invention, the degree of etherification is in a range of 0.5 to 0.9.

According to an embodiment of the present invention, the number average molecular weight is in a range of 50,000 to 80,000.

According to an embodiment of the present invention, the oxidized regenerated cellulose salt is a sodium salt, a calcium salt, a potassium salt, a magnesium salt or an aluminum salt.

According to an embodiment of the present invention, the hemostatic material exists in form of gel after absorbing water.

According to an embodiment of the present invention, the hemostatic material exists in form of power, fibre, woven fabric, non-woven fabric, sponge, film, hydrocolloid or foam.

In accordance with a second aspect of the present invention, a method for preparing a biodegradable and water-soluble hemostatic material comprises: oxidizing a regenerated cellulose with an oxidation system to prepare an oxidized regenerated cellulose having a degree of carboxylic acid oxidation not less than 5%; etherifying the oxidized regenerated cellulose to prepare an oxidized regenerated cellulose salt having a degree of etherification of 0.2 to 1.2 and a number average molecular weight of 50,000 to 200,000.

According to an embodiment of the present invention, the oxidation system is selected from a group consisting of a nitrogen oxide-type oxidation system based on NO₂ or N₂O₄, a nitroxide radical-type oxidation system based on TEMPO and a phosphoric acid solution of sodium nitrite or sodium nitrate.

According to an embodiment of the present invention, the step of oxidizing comprises: adding the regenerated cellulose in a solution of TEMPO and sodium bromide; adding a sodium hypochlorite solution into the solution of TEMPO and sodium bromide; adding a hydrochloric acid solution into the solution of TEMPO and sodium bromide until the solution of TEMPO and sodium bromide has a pH value of 10; and adding a sodium hydroxide solution into the solution of TEMPO and sodium bromide in order for keeping the pH value at 10.8 and generating the oxidized regenerated cellulose.

According to an embodiment of the present invention, the step of oxidizing further comprises: taking the oxidized regenerated cellulose out of the solution of TEMPO and sodium bromide and washing the oxidized regenerated cellulose with an ethanol solution having a concentration of 80% to 95%; and drying the oxidized regenerated cellulose.

According to an embodiment of the present invention, the step of etherifying comprises: adding the oxidized regenerated cellulose into an alkali solution in order for ageing the oxidized regenerated cellulose; adding an etherifying agent into the alkali solution in order for generating the oxidized regenerated cellulose salt; taking the oxidized regenerated cellulose salt out of the alkali solution and adding the oxidized regenerated cellulose salt into an acetic acid solution in order for neutralizing the oxidized regenerated cellulose salt; taking the oxidized regenerated cellulose salt out of the acetic acid solution, washing and drying; and packaging the oxidized regenerated cellulose salt and radiating the oxidized regenerated cellulose salt with an electron radiation for sterilization.

According to an embodiment of the present invention, the etherifying agent is a halogenated acid solution or a halogenated salt solution.

According to an embodiment of the present invention, the acetic acid solution has a concentration of 0.01 mol/L to 0.1 mol/L.

In the present invention, the hemostatic material has a reduced molecular weight of 50,000 to 200,000 through a selective oxidation process, thereby preventing formation of high molecular substances during hemostasia and risk of chronic poisoning resulted by the high molecular substances residing in human body. In addition, the hemostatic material has a degree of etherification of 0.2 to 1.2 through an etherification process, so that the hemostatic material has improved water solubility and can dissolves into flaky small molecular substances in a short period and can be absorbed by or excluded from human body.

### DETAILED DESCRIPTION OF PREFERRED EMBODIMENTS

The present invention provides a biodegradable and water-soluble hemostatic material which can offer improved hemostatic effect, absorbability and operability over existing hemostatic products. In the present invention, the hemostatic material is prepared from a regenerated cellulose, preferably from a viscose fibre having a degree of polymerization of 100 to 1,000, preferably 100 to 400. The regenerated cellulose can be selectively oxidized as an oxidized regenerated cellulose having a degree of carboxylic acid oxidation not less than 5%. Then, the oxidized regenerated cellulose is etherified as an oxidized regenerated cellulose salt having a degree of etherification of 0.2 to 1.2 and a number average molecular weight of 50,000 to 200,000.

In the present invention, the number average molecular weight of the oxidized regenerated cellulose salt is in a range of 50,000 to 200,000 and more preferably in a range of 50,000 to 80,000, so that the oxidized regenerated cellulose salt can be fully absorbed by or excluded from human body. The degree of carboxylic acid oxidation of the oxidized regenerated cellulose salt is not less than 5%, preferably in a range of 5% to 30%, more preferably in a range of 10% to 30%, and most preferably in a range of 18 to 24%, so that the hemostatic time can controlled in 2 minutes. In addition, the degree of etherification of the oxidized regenerated cellulose salt according to the present invention is in a range of 0.2 to 1.2, preferably in a range of 0.5 to 0.9, so that the hemostatic material can become transparent gel in 1 minute after contacting with body fluid and dissolve into small molecule substances and be absorbed by human body in a week.

The present invention further provides a method for preparing a biodegradable and water-soluble hemostatic material. The method mainly comprises following steps.

At first, a regenerated cellulose as raw material is oxidized by an oxidation system to generate an oxidized regenerated cellulose having a degree of carboxylic acid oxidation not less than 5%. In this step, viscose filament yarn gauze or viscose staple fiber gauze having a degree of polymerization of 100 to 400 is added into and oxidized by a nitrogen oxide-type oxidation system based on NO₂ or N₂O₄, a nitroxide radical-type oxidation system based on TEMPO or a phosphoric acid solution of sodium nitrite or sodium nitrate as a homogeneous oxidation system. The nitroxide radical-type oxidation system based on TEMPO is preferred in above-mentioned oxidation systems, and the oxidation process can comprise the steps of dissolving a quantity of TEMPO and sodium bromide in a water solution and adding the regenerated cellulose into the solution of TEMPO and sodium bromide. A sodium hypochlorite solution is added into the solution of TEMPO and sodium bromide. Then, a quantity of hydrochloric acid solution is added into the solution of TEMPO and sodium bromide until the solution of TEMPO and sodium bromide has a pH value of 10. Next, a sodium hydroxide solution is added into the solution of TEMPO and sodium bromide in order for keeping the pH value at 10.8. After reacting for a period of time, such as 80 to 240 minutes, the oxidized regenerated cellulose is taken out of the solution of TEMPO and sodium bromide and is washed by an ethanol solution which preferably has a concentration of 80% to 95%. The cleaned oxidized regenerated cellulose is dried.

Next, the oxidized regenerated cellulose prepared through above-mentioned processes is etherified to prepare an oxidized regenerated cellulose salt having a degree of etherification of 0.2 to 1.2 and a number average molecular weight of 50,000 to 200,000. In this embodiment, the oxidized regenerated cellulose salt can be a sodium salt, a calcium salt, a potassium salt, a magnesium salt or an aluminum salt. In particular, the etherification process comprises a step of adding the oxidized regenerated cellulose into an alkali solution which preferably is a sodium hydroxide solution or a calcium hydroxide solution having a concentration of 17% to 45% in order for ageing the oxidized regenerated cellulose. Following, an etherifying agent is added into the alkali solution to generate the oxidized regenerated cellulose salt from the oxidized regenerated cellulose. The etherifying agent preferably is a halogenated acid solution or a halogenated salt solution. After reacting for a period of time, the oxidized regenerated cellulose salt is taken out of the alkali solution, and added into an acetic acid(HAC) solution which preferably has a concentration of 0.01 mol/L to 0.1 mol/L in order for neutralizing the oxidized regenerated cellulose salt. Then, the neutralized oxidized regenerated cellulose salt is taken out of the acetic acid solution, cleaned and dried. The dried oxidized regenerated cellulose salt is packaged and radiated with an electron radiation for sterilization.

The biodegradable hemostatic material according to the present invention can become colorless and transparent gel after absorbing body fluid, which can cause minute stimulation to wound surfaces and can be observed easily, thereby providing protection to the wound surfaces. The biodegradable hemostatic material has a degree of etherification of 0.2 to 1.2 and a number average molecular weight of 50,000 to 200,000, and thus has an improved water-solubility. The biodegradable hemostatic material can dissolves into flaky small molecular substances in a shot period of time and can be fully absorbed by or excluded from human body.

The biodegradable hemostatic material according to the present invention can be widely used as hemostats or wound dressings for preventing wounds from sticking in clinical application. The biodegradable hemostatic material according to the present invention can be used in vitro or in vivo, and can be used in peripheral nerve surgery, oral surgery, nasal surgery, abdominal external surgery, thoracic surgery, digestive tract surgery, orthopaedic surgery and gynecologic surgery. The biodegradable hemostatic material according to the present invention can be absorbed by human body and can exist in form of power, fibre, woven fabric, non-woven fabric, sponge, film, hydrocolloid or foam. In particular, the hemostatic material can have a shape of sphere, column, plug or other suitable shapes which can cooperate with surgery instruments to function as hemostatic plugs for patients with coagulation dysfunction in tenestration surgery.

Furthermore, the hemostatic material according to the present invention can be prepared into gasoloid. At first, the hemostatic material is spray-dried or freeze-dried, and air-grinded or directly grinded into powers having a particle diameter less than 300 meshes. The powers can be combined with propulsorsors or surface active agents, such as pressurized nitrogen, carbon dioxide, freon, LPG or dimethyl ether, and drugs, and prepared together into the gasoloid. Alternatively, the powers can be spayed on wound surfaces by an electric or manual air pump for the purposes of hemostasis, absorbing exuding fluid, anti-inflammation, protecting wound surfaces and anti-sticking.

The hemostatic material according to the present invention can also be used with other biological products, drugs and agents, and can be used to prepare other medical materials or absorbable drug carriers which also belong to the hemostatic material of the present invention. The biological products, drugs and agents preferably include analgesics, anti-infective agents, antibiotics, anti-sticking agents, coagulants, and wound growth factors.

### EXAMPLE 1

10 g of viscose fibre gauze having a degree of polymerization of 100 to 400 is added into and oxidized by a nitroxide radical-type oxidation system based on TEMPO. In particular, 1,000 mg of TEMPO and 2.4 g of sodium bromide are dissolved in 1500 ml of water to form a solution of TEMPO and sodium bromide, and the gauze is added into the solution of TEMPO and sodium bromide. Then, 100 ml of sodium hypochlorite solution is added into the solution of TEMPO and sodium bromide. Then, a quantity of hydrochloric acid solution into the solution of TEMPO and sodium bromide until the solution of TEMPO and sodium bromide has a pH value of 10. Next, a sodium hydroxide solution having a concentration of 0.4 mol/L is added into the solution of TEMPO and sodium bromide in order for keeping the pH value at 10.8. After reacting for 80 minutes, the oxidized gauze is taken out of the solution of TEMPO and sodium bromide and is washed by an ethanol solution having a concentration of 85%. The cleaned oxidized gauze is dried.

The dried oxidized gauze is added into a sodium hydroxide solution having a concentration of 20%, and a halogenated acid solution as an etherifying agent is added into the sodium hydroxide solution to effect an etherification reaction for 1 hour. The etherified gauze is taken out of the sodium hydroxide solution and added into an acetic acid solution having a concentration of 0.05% for neutralizing the etherified gauze. The neutralized gauze is taken out of the acetic acid solution, cleaned, dried, packaged and radiated with an electron radiation for sterilization. The resultant gauze has a degree of carboxylic acid oxidation of 8% to 18%, a degree of etherification of 0.5 to 0.7, and a number average molecular weight of 70,000 to 80,000. The resultant gauze has improved water-solubility and suitable content of carboxylic acid, and can achieve hemostasis in 2 minutes and be absorbed by and excluded from human body in 1 to 2 weeks.

### EXAMPLE 2

10 g of viscose fibre gauze having a degree of polymerization of 100 to 400 is added into and oxidized by a nitroxide radical-type oxidation system based on TEMPO. In particular, 2000 mg of TEMPO and 4.8 g of sodium bromide are dissolved in 1500 ml of water to form a solution of TEMPO and sodium bromide, and the gauze is added into the solution of TEMPO and sodium bromide. Then, 200 ml of sodium hypochlorite solution is added into the solution of TEMPO and sodium bromide. Then, a quantity of hydrochloric acid solution is added into the solution of TEMPO and sodium bromide until the solution of TEMPO and sodium bromide has a pH value of 10. Next, a sodium hydroxide solution having a concentration of 0.4 mol/L is added into the solution of TEMPO and sodium bromide in order for keeping the pH value at 10.8. After reacting for 120 minutes, the oxidized gauze is taken out of the solution of TEMPO and sodium bromide and is washed by an ethanol solution having a concentration of 85%. The cleaned gauze is dried.

The dried gauze is added into a sodium hydroxide solution having a concentration of 30%, and a halogenated acid solution as an etherifying agent is added into the sodium hydroxide solution to effect an etherification reaction for 1 hour. The etherified gauze is taken out of the sodium hydroxide solution and added into an acetic acid solution having a concentration of 0.05% for neutralizing the etherified gauze. The neutralized gauze is taken out of the acetic acid solution, cleaned, dried, packaged and radiated with an electron radiation for sterilization. The resultant gauze has a degree of carboxylic acid oxidation of 18% to 24%, a degree of etherification of 0.7 to 0.9, and a number average molecular weight of 50,000 to 70,000. The resultant gauze has improved water-solubility and suitable content of carboxylic acid, and can achieve hemostasis in 2 minutes and be absorbed by and excluded from human body in 1 to 2 weeks.

### EXAMPLE 3

The resultant product of example 1 is dissolved in a water solution in a radio of 0.5% and the solution is low-temperature frozen and put into a freezing dryer to be freezing dried for 24 hours. The dried product is taken out of, packaged and radiated by an electron radiation, thereby forming a biodegradable hemostatic sponge. The hemostatic sponge has high flexibility, and can be used for hemostasis of various wound surfaces and has improved hemostatic effect.

### EXAMPLE 4

The resultant product of example 1 is prepared into gel in a radio of 60%. The gel is vacuum dried for 8 hours. The dried gel is grinded into particles having a diameter of 40 meshes. The particles are packaged and radiated by an electron radiation, thereby forming biodegradable hemostatic powders. The hemostatic powders can be used for hemostasis of various acute or chronic hemorrhages, and have improved hemostatic effect and portability.

The above-disclosed subject matter is to be considered illustrative, and not restrictive, and it will be apparent to those with ordinary skill in the art that various revisions and modifications are included in the present inventive concept, insofar as they do not depart from the spirit and scope of the present inventive concept. In accordance, the embodiments disclosed in the present inventive concept are intended to describe and not to restrict the technical scope of the present inventive concept, and therefore, the technical scope of the present inventive concept shall not be interpreted as being restricted in any way by the foregoing embodiments. Thus, to the maximum extent allowed by law, the scope of the present inventive concept is to be determined by the broadest permissible interpretation of the following claims and their equivalents, and shall not be restricted or limited by the foregoing detailed description.

What is claimed is:

1. A biodegradable and water-soluble hemostatic material, wherein the hemostatic material comprises an oxidized regenerated cellulose salt having a degree of carboxylic acid oxidation not less than 5%, a degree of etherification of 0.2 to 1.2 and a number average molecular weight of 50,000 to 200,000.

2. The hemostatic material of claim 1, wherein the hemostatic material is prepared from viscose fibre having a degree of polymerization of 100 to 1,000 through an oxidation process and an etherification process.

3. The hemostatic material of claim 1, wherein the degree of carboxylic acid oxidation is in a range of 18% to 24%.

4. The hemostatic material of claim 1, wherein the degree of etherification is in a range of 0.5 to 0.9.

5. The hemostatic material of claim 1, wherein the number average molecular weight is in a range of 50,000 to 80,000.

6. The hemostatic material of claim 1, wherein the oxidized regenerated cellulose salt is a sodium salt, a calcium salt, a potassium salt, a magnesium salt or an aluminum salt.

7. The hemostatic material of claim 1, wherein the hemostatic material exists in form of gel after absorbing water.

8. The hemostatic material of claim 1, wherein the hemostatic material exists in form of power, fibre, woven fabric, non-woven fabric, sponge, film, hydrocolloid or foam.

9. A method for preparing a biodegradable and water-soluble hemostatic material, wherein the method comprises:
oxidizing a regenerated cellulose with an oxidation system to prepare an oxidized regenerated cellulose having a degree of carboxylic acid oxidation not less than 5%;
etherifying the oxidized regenerated cellulose to prepare an oxidized regenerated cellulose salt having a degree of etherification of 0.2 to 1.2 and a number average molecular weight of 50,000 to 200,000.

10. The method of claim 9, wherein the oxidation system is selected from a group consisting of a nitrogen oxide-type oxidation system based on NO₂ or N₂O₄, a nitroxide radical-type oxidation system based on TEMPO and a phosphoric acid solution of sodium nitrite or sodium nitrate.

11. The method of claim 9, wherein the step of oxidizing comprises:
adding the regenerated cellulose in a solution of TEMPO and sodium bromide;
adding a sodium hypochlorite solution into the solution of TEMPO and sodium bromide;
adding a hydrochloric acid solution into the solution of TEMPO and sodium bromide until the solution of TEMPO and sodium bromide has a pH value of 10; and
adding a sodium hydroxide solution into the solution of TEMPO and sodium bromide in order for keeping the pH value at 10.8 and generating the oxidized regenerated cellulose.

12. The method of claim 11, wherein the step of oxidizing further comprises:
taking the oxidized regenerated cellulose out of the solution of TEMPO and sodium bromide and washing the oxidized regenerated cellulose with an ethanol solution having a concentration of 80% to 95%; and
drying the oxidized regenerated cellulose.

13. The method of claim 9, wherein the step of etherifying comprises:
adding the oxidized regenerated cellulose into an alkali solution in order for ageing the oxidized regenerated cellulose;
adding an etherifying agent into the alkali solution in order for generating the oxidized regenerated cellulose salt;
taking the oxidized regenerated cellulose salt out of the alkali solution and adding the oxidized regenerated cellulose salt into an acetic acid solution in order for neutralizing the oxidized regenerated cellulose salt;
taking the oxidized regenerated cellulose salt out of the acetic acid solution, washing and drying; and
packaging the oxidized regenerated cellulose salt and radiating the oxidized regenerated cellulose salt with an electron radiation for sterilization.

14. The method of claim 13, wherein the etherifying agent is a halogenated acid solution or a halogenated salt solution.

15. The method of claim 13, wherein the acetic acid solution has a concentration of 0.01 mol/L to 0.1 mol/L.
